(19) United States
(12) Patent Application Publication
(10) Pub. No.: US 2018/0160988 A1
(43) Pub. Date: Jun. 14, 2018

(54) Medical Alert Device

(71) Applicants: Liza Miller, Huntsville, AL (US); Brian Smith, Huntsville, AL (US)

(72) Inventors: Liza Miller, Huntsville, AL (US); Brian Smith, Huntsville, AL (US)

(21) Appl. No.: 15/377,219

(22) Filed: Dec. 13, 2016

(51) Int. Cl. A61B 5/00; H04B 1/3827; A61B 5/0205; G06F 19/00

(52) U.S. Cl. CPC A61B 5/746; H04B 1/385; A61B 5/6824; A61B 5/6831; A61B 5/7435; A61B 5/022; A61B 5/0004; G06F 19/3406; G06F 19/323; A61B 2560/0214; A61B 5/0205

(57) ABSTRACT

A medical alert device for measuring vitals and communicating with emergency response personnel includes a band that is configured to couple around a wrist of a user. An electronics module, a vitals module and a button are coupled to the band. The electronics module is configured to wirelessly communicate and to store a medical record of the user. The button and the vitals module are operationally coupled to the electronics module. The button is configured to be depressed to send a panic signal to the electronics module. The vitals module configured to assess at least one vital sign of the user. The vitals module is positioned to relay the at least one vital sign to the electronics module. The electronics module is configured to selectively wirelessly communicate the panic signal, the least one vital sign and the medical record to the emergency response personnel.

### CROSS-REFERENCE TO RELATED APPLICATIONS

Not Applicable

### STATEMENT REGARDING FEDERALLY SPONSORED RESEARCH OR DEVELOPMENT

Not Applicable

### THE NAMES OF THE PARTIES TO A JOINT RESEARCH AGREEMENT

Not Applicable

### INCORPORATION-BY-REFERENCE OF MATERIAL SUBMITTED ON A COMPACT DISC OR AS A TEXT FILE VIA THE OFFICE ELECTRONIC FILING SYSTEM

Not Applicable

### STATEMENT REGARDING PRIOR DISCLOSURES BY THE INVENTOR OR JOINT INVENTOR

Not Applicable

### BACKGROUND OF THE INVENTION

### (1) Field of the Invention

### (2) Description of Related Art Including Information Disclosed Under 37 CFR 1.97 and 1.98

The disclosure and prior art relates to alert devices and more particularly pertains to a new alert device for measuring vitals and communicating with emergency response personnel.

### BRIEF SUMMARY OF THE INVENTION

An embodiment of the disclosure meets the needs presented above by generally comprising a band that is configured to couple around a wrist of a user. An electronics module, a vitals module and a button are coupled to the band. The electronics module is configured to wirelessly communicate and to store a medical record of the user. The button and the vitals module are operationally coupled to the electronics module. The button is configured to be depressed to send a panic signal to the electronics module. The vitals module configured to assess at least one vital sign of the user. The vitals module is positioned to relay the at least one vital sign to the electronics module. The electronics module is configured to selectively wirelessly communicate the panic signal, the least one vital sign and the medical record to the emergency response personnel.

There has thus been outlined, rather broadly, the more important features of the disclosure in order that the detailed description thereof that follows may be better understood, and in order that the present contribution to the art may be better appreciated. There are additional features of the disclosure that will be described hereinafter and which will form the subject matter of the claims appended hereto.

The objects of the disclosure, along with the various features of novelty which characterize the disclosure, are pointed out with particularity in the claims annexed to and forming a part of this disclosure.

### BRIEF DESCRIPTION OF SEVERAL VIEWS OF THE DRAWING(S)

The disclosure will be better understood and objects other than those set forth above will become apparent when consideration is given to the following detailed description thereof. Such description makes reference to the annexed drawings wherein:

FIG. 1 is a front view of a medical alert device according to an embodiment of the disclosure.

FIG. 2 is a side view of an embodiment of the disclosure.

FIG. 3 is a detail view of an embodiment of the disclosure.

FIG. 4 is a cross-sectional view of an embodiment of the disclosure.

FIG. 5 is an isometric perspective view of an embodiment of the disclosure.

### DETAILED DESCRIPTION OF THE INVENTION

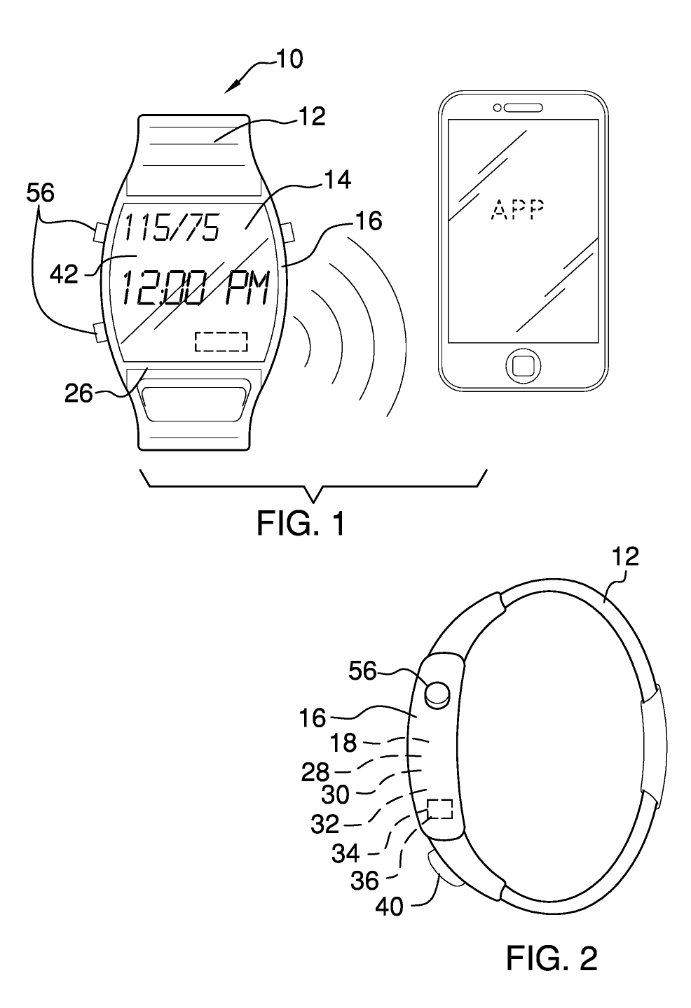

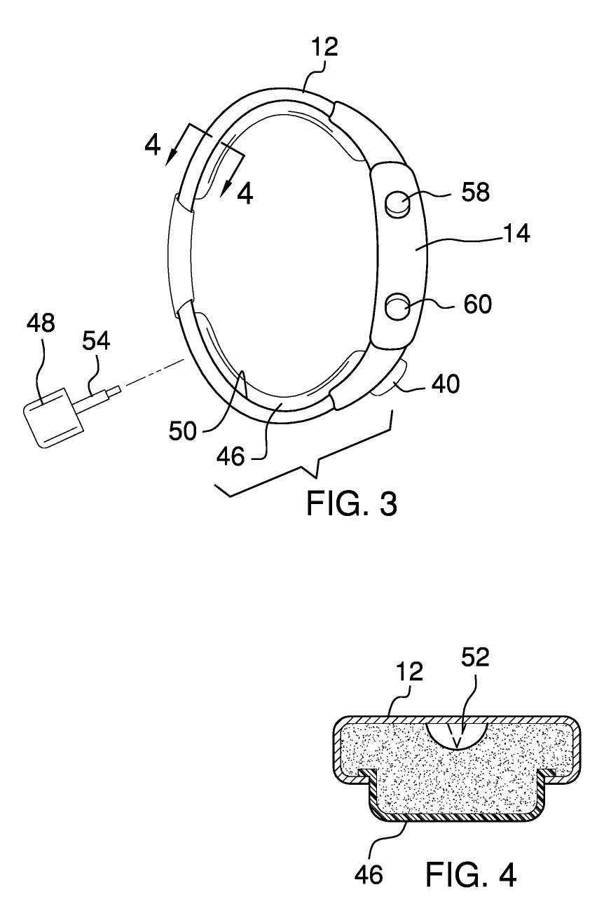

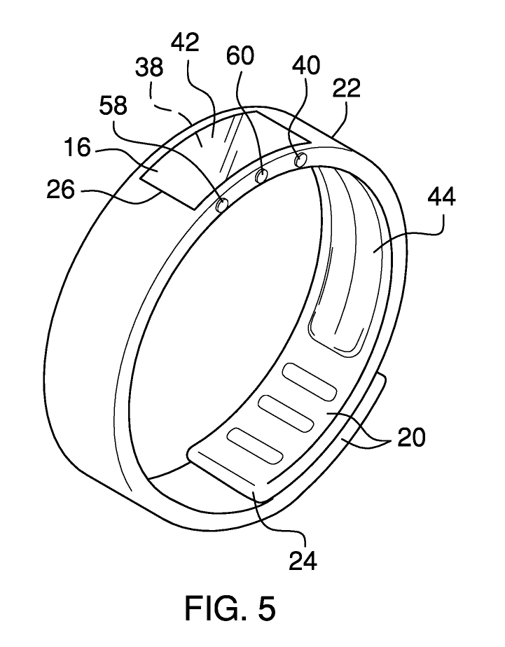

With reference now to the drawings, and in particular to FIGS. 1 through 5 thereof, a new alert device embodying the principles and concepts of an embodiment of the disclosure and generally designated by the reference numeral 10 will be described.

As best illustrated in FIGS. 1 through 5, the medical alert device 10 generally comprises a band 12 that is configured to couple around a wrist of a user. An electronics module 14 is coupled to the band 12. The electronics module 14 is configured to wirelessly communicate. The electronics module 14 also is configured to store a medical record of the user. In one embodiment, the electronics module 14 comprises a housing 16 that defines an internal space 18. In another embodiment, the band 12 comprises a pair of arms 20. Each arm 20 has a first end 22 and a second end 24. The first end 22 is flexibly coupled to the housing 16. Each arm 20 extends from a respective opposing end 26 of the housing 16 such that the second ends 24 are overlapping. The arms 20 are positioned on the housing 16 such that the arms 20 are flexibly positionable and the band 12 is configured to position around the wrist of the user.

A power module 28, a microprocessor 30, a data storage module 32, a transmitter 34 and a receiver 36 are coupled to the housing 16 and are positioned in the internal space 18. In one embodiment, the power module 28 comprises at least one battery 38. The microprocessor 30 is operationally coupled to the power module 28. The data storage module 32, the transmitter 34 and the receiver 36 are operationally coupled to the microprocessor 30. The data storage module 32 is configured to store the medical record of the user. The receiver 36 is global positioning system enabled. The receiver 36 is configured to receive location coordinates of the user upon depression of a button 40. The receiver 36 is positioned to relay the location coordinates to the microprocessor 30. The microprocessor 30 is positioned to compel the transmitter 34 to selectively wirelessly communicate a panic signal, at least one vital sign, the medical record and the location coordinates to emergency response personnel.

A screen 42 is coupled to the housing 16. The screen 42 is operationally coupled to the microprocessor 30. The microprocessor 30 is positioned to compel the screen 42 to selectively display a time and the at least one vital sign of the user. In one embodiment, the screen 42 is touch enabled.

The button 40 is coupled to the band 12 and is operationally coupled to the electronics module 14. The button 40 is depressible. The button 40 is configured to be depressed to send the panic signal to the electronics module 14. The electronics module 14 is configured to wireles sly communicate the panic signal to the emergency response personnel.

A vitals module 44 is coupled to the band 12. The vitals module 44 is operationally coupled to the electronics module 14. The vitals module 44 is configured to assess the at least one vital sign of the user. The vitals module 44 is positioned to relay the at least one vital sign to the electronics module 14. The electronics module 14 is configured to wirelessly communicate the least one vital sign to the emergency response personnel.

In one embodiment, the vitals module 44 comprises a bladder 46 and a pump 48. The bladder 46 is coupled to an inside face 50 of the band 12. The bladder 46 is selectively inflatable such that the bladder 46 is selectively extensible from the inside face 50 of the band 12. The pump 48 is operationally coupled to the bladder 46. The bladder 46 is positioned to selectively inflate the bladder 46 such that the bladder 46 is configured to contact the wrist of the user.

In another embodiment, a valve 52 is coupled to the band 12. The valve 52 is fluidically coupled to the bladder 46. The valve 52 is positioned on the band 12 such that the valve 52 is positioned to couple to a stem 54 of the pump 48. The pump 48 is reversibly fluidically couplable to the bladder 46. The bladder 46 is positioned between the inside face 50 of the band 12 and the wrist of the user such that the bladder 46 is configured to assess and relay a pulse rate and a blood pressure to the electronics unit.

A control module 56 is coupled to the housing 16. The control module 56 is operationally coupled to the microprocessor 30. The control module 56 is configured to enter commands into the microprocessor 30. In one embodiment, the control module 56 comprises a first knob 58 and a second knob 60. The first knob 58 and the second knob 60 are depressible. The first knob 58 is positioned to be depressed to signal the microprocessor 30 to display the at least one vital sign. The second knob 60 is positioned to be depressed to signal the microprocessor 30 to compel the screen 42 to display a setup menu. The screen 42 is configured to input commands into the microprocessor 30.

In use, the button 40 is configured to be depressed to send the panic signal to the electronics module 14. The receiver 36 is configured to receive location coordinates of the user upon depression of the button 40. The receiver 36 is positioned to relay the location coordinates to the microprocessor 30. The microprocessor 30 is positioned to compel the transmitter 34 to selectively wirelessly communicate the panic signal and the location coordinates to the emergency response personnel. The bladder 46 is positioned between the inside face 50 of the band 12 and the wrist of the user such that the bladder 46 is configured to assess and relay the pulse rate and the blood pressure to the microprocessor 30. The microprocessor 30 is positioned to compel the transmitter 34 to selectively wirelessly communicate the pulse rate and the blood pressure to the emergency response personnel.

With respect to the above description then, it is to be realized that the optimum dimensional relationships for the parts of an embodiment enabled by the disclosure, to include variations in size, materials, shape, form, function and manner of operation, assembly and use, are deemed readily apparent and obvious to one skilled in the art, and all equivalent relationships to those illustrated in the drawings and described in the specification are intended to be encompassed by an embodiment of the disclosure.

Therefore, the foregoing is considered as illustrative only of the principles of the disclosure. Further, since numerous modifications and changes will readily occur to those skilled in the art, it is not desired to limit the disclosure to the exact construction and operation shown and described, and accordingly, all suitable modifications and equivalents may be resorted to, falling within the scope of the disclosure. In this patent document, the word “comprising” is used in its non-limiting sense to mean that items following the word are included, but items not specifically mentioned are not excluded. A reference to an element by the indefinite article “a” does not exclude the possibility that more than one of the element is present, unless the context clearly requires that there be only one of the elements.

I claim:

1. Medical alert device comprising:
a band configured for coupling around a wrist of a user;
an electronics module coupled to said band, said electronics module being configured for wireless communication, said electronics module being configured for storage of a medical record of the user;
a button coupled to said band, said button being operationally coupled to said electronics module, said button being depressible;
a vitals module coupled to said band, said vitals module being operationally coupled to said electronics module; and
wherein said button is positioned on said band such that said button is configured for depressing for sending a panic signal to said electronics module, wherein said vitals module is positioned on said band such that said vitals module is configured for assessing at least one vital sign of the user, wherein said vitals module is positioned for relaying the at least one vital sign to said electronics module, wherein said electronics module is configured for selectively wireles sly communicating the panic signal, the least one vital sign and the medical record to emergency response personnel.

2. The device of claim 1, further including said band comprising a pair of arms, each said arm having a first end and a second end, said first end being flexibly coupled to said housing, each said arm extending from a respective opposing end of said housing such that said second ends are overlapping, wherein said arms are positioned on said housing such that said arms are flexibly positionable such that said band is configured for positioning around the wrist of the user.

3. The device of claim 1, further including said electronics module comprising:
a housing defining an internal space;
a power module coupled to said housing and positioned in said internal space;
a microprocessor coupled to said housing and positioned in said internal space; said microprocessor being operationally coupled to said power module,
a data storage module coupled to said housing and positioned in said internal space, said data storage module being operationally coupled to said microprocessor, said data storage module being configured for storage of the medical record of the user;
a transmitter coupled to said housing and positioned in said internal space, said transmitter being operationally coupled to said microprocessor; and
wherein said microprocessor is positioned to compel said transmitter to selectively wirelessly communicate the least one vital sign to the emergency response personnel, wherein said microprocessor is positioned to compel said transmitter to selectively wireles sly communicate the medical record of the user to the emergency response personnel.

4. The device of claim 1, further including said power module comprising at least one battery.

5. The device of claim 1, further including a screen coupled to said housing, said screen being operationally coupled to said microprocessor, wherein said microprocessor is positioned to compel said screen to selectively display a time and the at least one vital sign of the user.

6. The device of claim 1, further including said screen being touch enabled.

7. The device of claim 1, further including a receiver coupled to said housing and positioned in said internal space, said receiver being global positioning system enabled, said receiver being operationally coupled to said microprocessor, wherein said receiver is positioned in said housing such that said receiver is configured for receiving location coordinates of the user upon depression of said button, such that said receiver is positioned to relay the location coordinates to said microprocessor, wherein said microprocessor is positioned to compel said transmitter to selectively wirelessly communicate the location coordinates to the emergency response personnel.

8. The device of claim 1, further including said vitals module comprising:
a bladder coupled to an inside face of said band, said bladder being selectively inflatable such that said bladder is selectively extensible from said inside face of said band;
a pump operationally coupled to said bladder; and
wherein said bladder is positioned for selectively inflating said bladder such that said bladder is configured for contacting the wrist of the user, wherein said bladder is positioned between said inside face of said band and the wrist of the user such that said bladder is configured for assessing and relaying a pulse rate and a blood pressure to said electronics unit.

9. The device of claim 8, further including a valve coupled to said band, said valve being fluidically coupled to said bladder, wherein said valve is positioned on said band such that said valve is positioned for coupling to a stem of said pump, such that said pump is reversibly fluidically couplable to said bladder.

10. The device of claim 1, further including a control module coupled to said housing, said control module being operationally coupled to said microprocessor, wherein said control module is coupled to said microprocessor such that said control module is configured for entering of commands into said microprocessor.

11. The device of claim 10, further including said control module comprising a first knob and a second knob, said first knob and said second knob being depressible, wherein said first knob is positioned on said housing such that said first knob is positioned for depressing for signaling said microprocessor to display the at least one vital sign and wherein said second knob is positioned on said housing such that said second knob is positioned for depressing for signaling said microprocessor to compel said screen to display a setup menu, such that said screen is configured for inputting commands into said microprocessor.

12. Medical alert device comprising:
a band configured for coupling around a wrist of a user;
an electronics module coupled to said band, said electronics module being configured for wireless communication, said electronics module being configured for storage of a medical record of the user, said electronics module comprising:
a housing defining an internal space,
a power module coupled to said housing and positioned in said internal space, said power module comprising at least one battery,
a microprocessor coupled to said housing and positioned in said internal space, said microprocessor being operationally coupled to said power module,
a data storage module coupled to said housing and positioned in said internal space, said data storage module being operationally coupled to said microprocessor, said data storage module being configured for storage of the medical record of the user,
a transmitter coupled to said housing and positioned in said internal space, said transmitter being operationally coupled to said microprocessor, wherein said microprocessor is positioned to compel said transmitter to selectively wirelessly communicate a panic signal, at least one vital sign and the medical record to emergency response personnel, and
a screen coupled to said housing, said screen being operationally coupled to said microprocessor, wherein said microprocessor is positioned to compel said screen to selectively display a time and the at least one vital sign of the user, said screen being touch enabled;
a button coupled to said band, said button being operationally coupled to said electronics module, said button being depressible, wherein said button is positioned on said band such that said button is configured for depressing for sending a panic signal to said electronics module, wherein said electronics module is configured for wirelessly communicating the panic signal to the emergency response personnel;
a receiver coupled to said housing and positioned in said internal space, said receiver being global positioning system enabled, said receiver being operationally coupled to said microprocessor, wherein said receiver is positioned in said housing such that said receiver is configured for receiving location coordinates of the user upon depression of said button, such that said receiver is positioned to relay the location coordinates to said microprocessor, wherein said microprocessor is positioned to compel said transmitter to selectively wirelessly communicate the location coordinates to the emergency response personnel;
a vitals module coupled to said band, said vitals module being operationally coupled to said electronics module, wherein said vitals module is positioned on said band such that said vitals module is configured for assessing the at least one vital sign of the user, wherein said vitals module is positioned for relaying the at least one vital sign to said electronics module, wherein said electronics module is configured for wirelessly communicating the least one vital sign to the emergency response personnel, said vitals module comprising:
a bladder coupled to an inside face of said band, said bladder being selectively inflatable such that said bladder is selectively extensible from said inside face of said band,
a pump operationally coupled to said bladder, wherein said bladder is positioned for selectively inflating said bladder such that said bladder is configured for contacting the wrist of the user,
a valve coupled to said band, said valve being fluidically coupled to said bladder, wherein said valve is positioned on said band such that said valve is positioned for coupling to a stem of said pump, such that said pump is reversibly fluidically couplable to said bladder, and
wherein said bladder is positioned between said inside face of said band and the wrist of the user such that said bladder is configured for assessing and relaying a pulse rate and a blood pressure to said electronics unit;
a control module coupled to said housing, said control module being operationally coupled to said microprocessor, wherein said control module is coupled to said microprocessor such that said control module is configured for entering of commands into said microprocessor, said control module comprising a first knob and a second knob, said first knob and said second knob being depressible, wherein said first knob is positioned on said housing such that said first knob is positioned for depressing for signaling said microprocessor to display the at least one vital sign and wherein said second knob is positioned on said housing such that said second knob is positioned for depressing for signaling said microprocessor to compel said screen to display a setup menu, such that said screen is configured for inputting commands into said microprocessor; and
wherein said button is positioned on said band such that said button is configured for depressing for sending a panic signal to said electronics module, wherein said receiver is positioned in said housing such that said receiver is configured for receiving location coordinates of the user upon depression of said button, such that said receiver is positioned to relay the location coordinates to said microprocessor, wherein said microprocessor is positioned to compel said transmitter to selectively wirelessly communicate the panic signal and the location coordinates to the emergency response personnel, wherein said bladder is positioned between said inside face of said band and the wrist of the user such that said bladder is configured for assessing and relaying a pulse rate and a blood pressure to said microprocessor, wherein said microprocessor is positioned to compel said transmitter to selectively wireles sly communicate the pulse rate and the blood pressure to the emergency response personnel.

13. The device of claim 12, further including said band comprising a pair of arms, each said arm having a first end and a second end, said first end being flexibly coupled to said housing, each said arm extending from a respective opposing end of said housing such that said second ends are overlapping, wherein said arms are positioned on said housing such that said arms are flexibly positionable such that said band is configured for positioning around the wrist of the user.
